(19) United States
(12) Patent Application Publication
(10) Pub. No.: US 2008/0020043 A1
(43) Pub. Date: Jan. 24, 2008

(54) Dendrimer-Drug Conjugates

(76) Inventors: Marc Gingras, St. Laurent du Var (FR); Jean-Manuel Raimundo, Tourrettes-Levens (FR); Yoann M. Chabre, Nice (FR); Michele Boix, Cantaron (FR); Marina Do, Menton (FR); Christian Sarrazin, Pegomas (FR); Laurent Delahaye, Villeuneuve Loubet (FR)

Correspondence Address: Robert J. Baran (T2-7H);ALLERGAN, INC., Legal Department, 2525 Dupont Drive, Irvine, CA, 92612, US

(21) Appl. No.: 11/458,901

(22) Filed: Jul. 20, 2006

(51) Int. Cl. A61K 9/00

(52) U.S. Cl. 424/486

(57) ABSTRACT

The present invention provides a drug-dendrimer conjugate comprising a central core, two or more linear hydrophilic molecules bonded thereto, a secondary core molecule bonded to at least a majority, if not all, of said first linear hydrophilic molecules, a drug or other biologically-active molecule bonded to the remainder of said first linear hydrophilic molecules or bonded to a segment comprising a second secondary core molecule which is bonded to said first secondary core molecule by a linear hydrophilic molecule, and wherein at least some of the bonds between said drug or other biologically active molecule and said first and/or second secondary core molecules are hydrolysable by an endogenous esterase.

### BACKGROUND OF THE INVENTION

1. Field of the Invention

The present invention relates to combination of drugs or other biologically-active molecules covalently bonded to dendrimers, i.e. dendrimer-drug conjugates.

2. Background of the Art

Dendrimer synthesis is a relatively new field of polymer chemistry defined by regular, highly branched monomers leading to a monodisperse, tree-like or generational structure. Synthesizing monodisperse polymers demands a high level of synthetic control which is achieved through stepwise reactions, building the dendrimer up one monomer layer, or “generation,” at a time. Each dendrimer consists of a multifunctional core molecule with a dendritic wedge attached to each functional site. The core molecule is referred to as “generation 0.” Each successive repeat unit along all branches forms the next generation, “generation 1,” “generation 2,” and so on until the terminating generation.

There are two defined methods of dendrimer synthesis, divergent and convergent. In the divergent method, the molecule is assembled from the core to the periphery; while in the convergent method, the dendrimer is synthesized beginning from the outside and terminating at the core. In either method the synthesis requires a stepwise process, attaching one generation to the last, purifying, and then changing functional groups for the next stage of reaction.

This functional group transformation is necessary to prevent unbridled polymerization. Such polymerization would lead to a highly branched molecule which is not monodisperse—otherwise known as a hyperbranched polymer.

In the divergent method, the surface groups initially are unreactive or protected species which are converted to reactive species for the next stage of the reaction. In the convergent approach the opposite holds, as the reactive species must be on the focal point of the dendritic wedge.

Due to steric effects, continuing to react dendrimer repeat units leads to a sphere shaped or globular molecule until steric overcrowding prevents complete reaction at a specific generation and destroys the molecule's monodispersity. The number of possible generations can be increased by using longer spacing units in the branches of the core molecule. The monodispersity and spherical steric expansion of dendrimers leads to a variety of interesting properties.

The steric limitation of dendritic wedge length leads to small molecular sizes, but the density of the globular shape leads to fairly high molecular weights. The spherical shape also provides an interesting study in molecular topology. Dendrimers have two major chemical environments, the surface chemistry due to the functional groups on the termination generation which is the surface of the dendritic sphere, and the sphere's interior which is largely shielded from exterior environments due to the spherical shape of the dendrimer structure. The existence of two distinct chemical environments in such a molecule implies many possibilities for dendrimer applications.

Theoretically, hydrophobic/hydrophilic and polar/nonpolar interactions can be varied in the two environments. The existence of voids in the dendrimer interior furthers the possibilities of these two heterogeneous environments playing an important role in dendrimer chemistry. Dendrimer research has confirmed the ability of dendrimers to accept guest molecules in the dendritic voids.

Dendrimers have found actual and potential use as molecular weight and size standards, gene transfection agents, as hosts for the transport of biologically important guests, and as anti-cancer agents, to name but a few. Much of the interest in dendrimers involves their use as catalytic agents, utilizing their high surface functionality and ease of recovery. Dendrimers' globular shape and molecular topology, however, make them highly useful to biological systems as well.

Combination of dendrimers and drugs or other biologically active molecules are disclosed in the following patents and applications which are hereby incorporated by reference in their entirety for the purpose of showing how to make and use the drug-dendrimer conjugates of the present invention:

U.S. Pat. Nos. 5,714,166; 6,585,956; 6,020,457; 6,225,352; 4,599,400; 5,567,411; 6,664,315; 6,037,444; 6,300,424; 5,648,506; 6,417,339; 6,632,889; U.S. Publication Nos. 2003-064050; 2002-054863; 2003-023968; 2003-211072; 2002-123609; and PCT Publication WO/003923.

Not withstanding the above, no patent specifically claims (and no publication specifically describes) a macromolecular globular dendritic structure for controlled delivery with terminal functionalities containing drugs or other biologically-active molecules, which can be released by enzymatic cleavage.

### BRIEF SUMMARY OF THE INVENTION

1. The present invention provides a dendrimer-drug conjugate comprising the following elements;

(a) a central core molecule having at least two, and preferably three or more, functional groups capable of reacting with a first linear hydrophilic molecule to form a covalent bond between said central core molecule and said first linear hydrophilic molecule;

(b) a first linear hydrophilic molecule capable of reacting with said central core molecule to form a first segment of said conjugate comprising multiple branches of said first linear hydrophilic molecule emanating from said central core molecule, wherein said first segment of said conjugate comprises a covalent bond between said central core molecule and said first linear hydrophilic molecule and wherein said linear hydrophilic molecule comprises at least one additional functional group capable of reacting with a secondary core molecule to form a covalent bond between said first linear hydrophilic molecule and said secondary core molecule;

(c) a secondary core molecule having at least one functional group capable of reacting with said additional functional group of said first linear hydrophilic molecule to form a covalent bond between said secondary core molecule and said first linear hydrophilic molecule and having at least two additional functional groups capable of reacting with a second linear hydrophilic molecule;

(d) a second linear hydrophilic molecule having a functional group capable of reacting with said additional functional groups of said secondary core molecule to form a second segment of said conjugate comprising multiple branches of said second linear hydrophilic molecule emanating from said secondary core molecule, wherein said second segment of said conjugate comprises a covalent bond between said secondary core molecule and said second linear hydrophilic molecule and wherein said second linear hydrophilic molecule has at least one additional functional group capable of reacting with a drug or other biologically-active molecule; and

(e) one or more drugs or other biologically active molecules capable of reacting with said additional functional group of said second linear hydrophilic molecule to provide said dendrimer-drug conjugate.

As noted above, in describing dendrimers, the drug-dendrimer conjugate comprises a central core molecule (generation 0), a first linear hydrophilic molecule (generation 1), a secondary core molecule (generation 3), a second linear hydrophilic molecule (generation 4) and a drug or other biologically active molecule (generation 5).

Alternatively, said secondary core molecule may be bonded directly to said central core through a covalent bond.

In another embodiment of the invention, the drug-dendrimer has a segment comprising a second secondary core molecule and a third linear hydrophilic molecule to provide a third segment inserted between said second linear hydrophilic molecule of element (d) above, (or said second segment) and said biologically active molecule. Thus, in this embodiment, as compared to the drug-dendrimer conjugate described above, the drug-dendrimer conjugate has 7 generations.

Alternatively, said third segment may comprise a second secondary core molecule bonded directly to said first secondary core molecule.

In a further embodiment of the present invention, the drug-dendrimer conjugate comprises two or more different biologically-active molecules. In this embodiment, a first biologically-active molecule may be bonded to an earlier generation than a second biologically active molecule. For example, a first biologically-active molecule may be bonded to said first secondary core molecule and a second biologically-active molecule may be bonded to said second secondary core molecule.

Preferably said covalent bond of element (d) of said dendrimer-drug is hydrolyzed in the presence of an endogenous esterase.

The esterase hydroyzable covalent bond may be selected from the group consisting of an ester, an amide, a carbonate, a carbamate, a urea bond and mixtures thereof.

In the drug-dendrimer of the present invention, either or both of said first linear hydrophilic molecule and said second linear hydrophilic molecule may be a polyoxyethylene molecule.

Furthermore, in the drug-dendrimer conjugate of the invention, either or all of said central core molecule and said secondary core molecules may be a polyhydroxy organic compound.

### DESCRIPTION OF THE DRAWINGS FIGURES

FIG. 1 represents a dendrimer-drug conjugate according to the invention wherein two biologically-active substances are covalently bonded to a dendrimer-like structure.

FIG. 2 represents a dendrimer-drug conjugate according to the invention wherein a secondary core is bonded to a central core through a hydrophilic segment or an enzyme degradable linkage and a biologically-active substance is bonded to said secondary core.

FIG. 3 represents a dendrimer-drug conjugate according to the invention wherein a first secondary core is bonded to a central core, a second secondary core is bonded to said first secondary core and a biologically active compound is bonded to said second secondary core.

FIG. 4 represents a dentrimer-drug conjugate according to the invention (Structure IV) and various conjugate substructures which release drug more quickly than the dendrimer-drug conjugate of the invention. (See Structures II and III.)

FIG. 5 represents a dendrimer-drug conjugate according to the invention wherein one or more biologically active compounds are bonded in either, or both, of a internal layer or generation and an external layer or generation.

FIG. 6 represents a dendrimer-drug conjugate according to the invention wherein a first secondary core is bonded to a central core, a second secondary core is bonded to said first secondary core and a biologically active compound is bonded to said second secondary core.

FIG. 7 shows an HPLC chromatogram of flurbiprofen, a test drug, alone, and bonded to Structures II and III of FIG. 4.

FIG. 8 shows the results of enzymatically-hydrolyzing a drug conjugate comprising flurbiprofen, a test drug and Structures II and III of FIG. 4.

FIG. 9 shows the various embodiments of the dendrimer-drug conjugates according to the invention and intermediates useful in the preparation thereof.

FIG. 10 describes a scheme for the synthesis of the conjugate comprising flurbiprofen, a test compound, and a dendrimer.

### DETAILED DESCRIPTION OF THE INVENTION

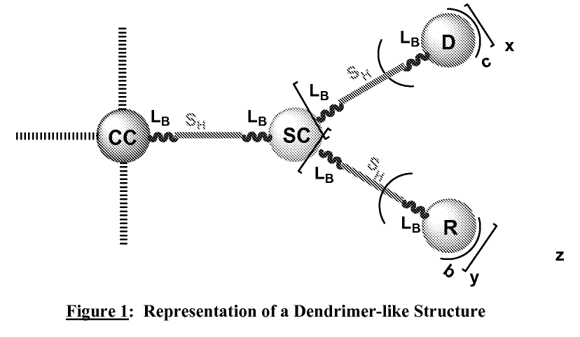

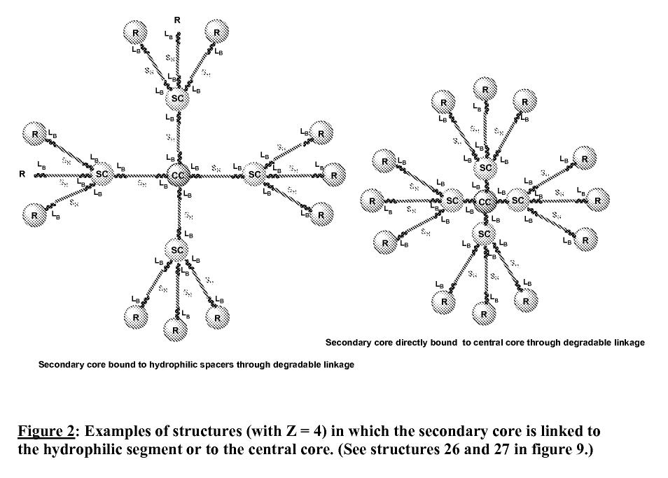

The invention is directed to the preparation of “dendrimer-like” structures as shown in FIG. 1 to allow a sustained delivery of drugs, wherein said drug is covalently attached to the dendrimer carrier molecule by enzymatically degradable linkages such as ester, amide, carbonate, or urethane linkages. It is possible to provide variations in the molecular architecture of the structure by means of step-wise chemical synthesis of a central core or generation 0, a secondary core, or generation 1 and an additional hydrophilic segment or generation 3.

These variations will provide for the drug delivery system (DDS) properties.

Drugs, or other biologically active molecules, including but not limited to peptides, proteins, enzymes, small molecules drugs (antibiotics, fungicides, anti-viral, anti-inflammatory, anti-tumor, cardiovascular, etc . . . ), dyes, lipids, nucleosides, etc., may be included in the dendrimer-drug conjugates of the invention. The solubility of a large molecule such as the dendrimer-drug conjugate is achieved through incorporation of hydrophilic spacers, e.g. poly(ethylene glycol) (PEG) via enzyme-degradable or hydrolyzable linkages in such way they form a hydrophilic layer at different levels of the final chemical structure of the dentrimer-drug conjugate of the invention.

In the dendrimer-drug conjugate shown in FIG. 1:
CC represents the central core bonded to dendron z
SC represents the secondary core which may be identical or different from CC;
z is ≧1, x is ≧1, y is ≧1 and x=y or x≠y, provided that at least one of x, y or z must be >1;
D represents a drug or biologically active substance, or
if D is not a drug or other biologically active substance,
D may be Z or SC (which may be different or identical to the Z or SC previously defined) or any combination of SC, CC, L_{B}, S_{H}, D or R;
R represents a drug or biologically active substance which may be identical to or different then D, or if R is not a drug or other biologically active substance,
R may be Z or SC (which may be different or identical to the Z or SC previously defined) or any combination of SC, CC, L_{B}, S_{H}, D, R;
L_{B} represents biodegradable linkages which may be different or not; and
S_{H} represents hydrophilic chemically defined spacers which may be different or not and which may be linear or not; and b and c≧1 and b and c may be the same or different.

The central core (CC) is a reactive core allowing several branch lines to be formed. Preferably CC is selected from the group consisting of an aliphatic, cycloaliphatic or aromatic alcohol, a diol, a triol (e.g. phloroglucinol), a tetrol (e.g. pentaerythritol), a reducing sugar e,g, sorbitol, mannitol, arabitol, glycerol dipentaerythrytol, glycerol oligomers (hexaglycerol), a synthesized polyol, a thiol or polythiol, a polyamine, a halomethylaryl compound represented by the formula

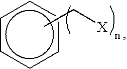

a acid halide (e.g. a aromatic or aliphatic acid halide such as

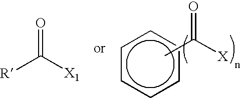

wherein R′ is an aliphatic radical, n is an integer of from 2 to 6 and X is Br, Cl, I, or another leaving group;
or any other structure built by the combination of one or more of the above molecules

The secondary core (SC) may be identical or different than CC:

Preferably SC is selected from the group consisting of monomers of formula A-R′—B₂, wherein R′ is an aromatic (phenyl, naphthyl . . . ), or aliphatic radical, A and B are functional groups capable of forming a covalent bond with either a preceding or subsequent generation of the drug-dendrimer conjugate and preferably selected to provide that only one group (A or B) can react first whereas the second one does not react or remains protected and monomers of formula A-R″—B₃ wherein R″ is an aromatic radical and A and B are as defined above.

Example of secondary cores A-R′—B₂ used in this invention include:

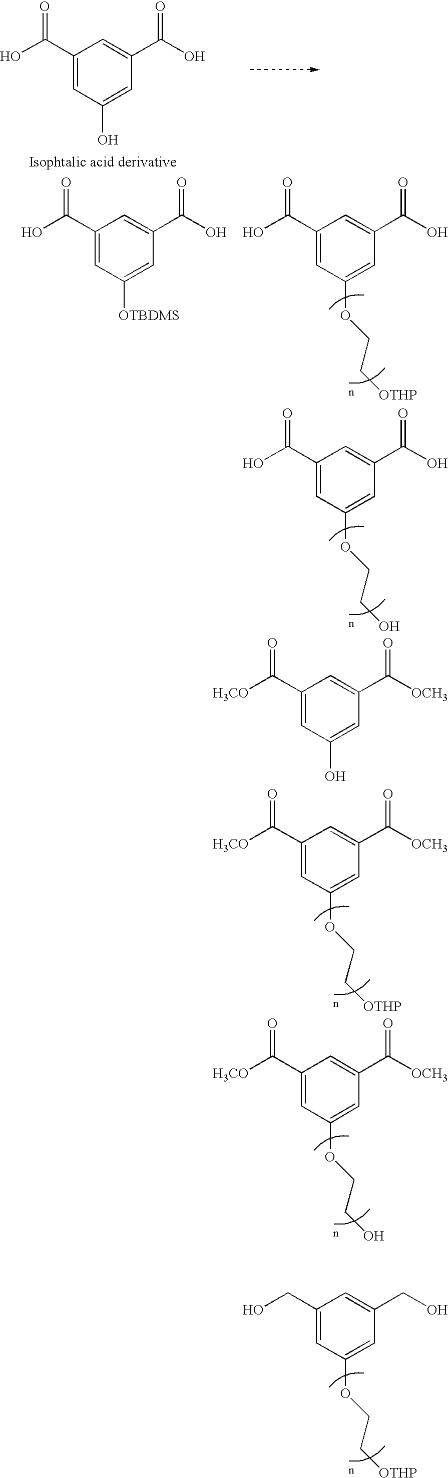

wherein TBDMS is t-butyl dimethyl silyl and THP is tetrahydropyran.

Examples of secondary cores A-R″—B3 used in this invention include:

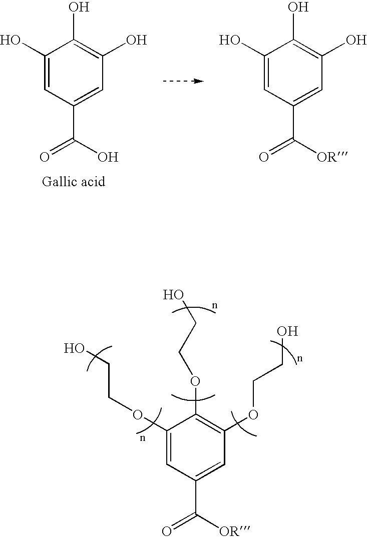

wherein R′″ is alkyl.

The secondary core can be bonded, through an enzyme-degradable chemical linkage, to the central core or to hydrophilic segment as shown in FIG. 2, wherein CC, SC, R, Z, L_{B} and S_{H} are as defined above.

In order to allow the dentrimer-drug conjugates of the invention to exhibit satisfactory water solubility, a hydrophilic chemically defined spacer is incorporated in the dentrimer-drug conjugate providing that said spacer presents low toxicity and is a biocompatible polymer (including linear or non-linear polymers) such as: (poly(ethylene glycol) (PEG), PEG-like spacers, poly(amino acid) (linear poly(lysine), polyvinyl alcohol, polyhydroxyethylmethacrylate, polyacrylamide, polyacrylic acid, polyethyloxazoline, polyvinyl pyrrolidinone, polysaccharides such as agarose, chitosan, alginates, hyaluronic acid, dextrans, etc. and it brings no polydispersity to the final structure which is a critical factor to ensure the synthesis of the preferred drug-dendrimer conjugate of this invention.

For example, preferably linear poly(ethylene glycol) (PEG) is used as the spacer.

The PEG spacer may include different M end-capped groups like amino, ester, carboxylic acid, succinic acid, amide, urethane, thiol, etc . . . (in place of the hydroxyl functionality) to allow further diversity and variability in the molecular construction.

For example, the PEG spacer utilized in the present invention may include different end-capped groups as illustrated below.

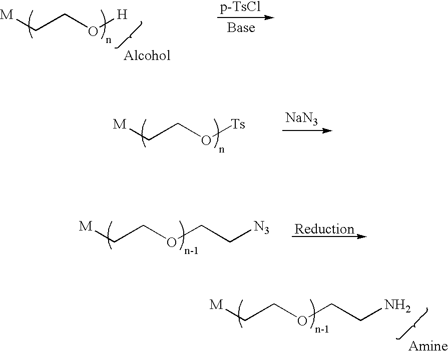

Ts is tosyl, M, in this illustrative chemical synthesis scheme, may be D, R, CC, SC, —OH, —SH, —NH₂, protecting groups, carboxylic acid, amide, ester, urethane, etc. and n is an integer of from 1 to 7.

Higher molecular weight monodisperse PEG or PEG-like spacers can be obtained by using an iterative process by addition of commercial or modified monodisperse PEG units. PEG monodispersity is controlled by using a chain length (n) of from 1 to 7.

For example, in the following reaction scheme

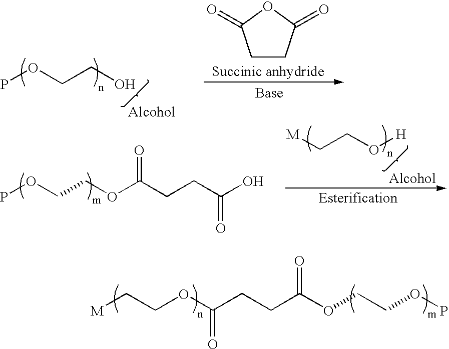

wherein M and n are as defined above;

P represents protecting groups and m is an integer of from 1 to 7.

The spacer is covalently attached to the structure (“interior”) of the dendrimer structure and to the drug through a degradable linkage moiety, e.g. an enzyme esterase, i.e. a hydrolase.

Spacers are incorporated in the dendrimer structure in such way to form a hydrophilic layer which can be present at different levels in the structure. Thus, in case of their presence at several levels in the structure, they are distributed into successive or different hydrophilic layers or generations.

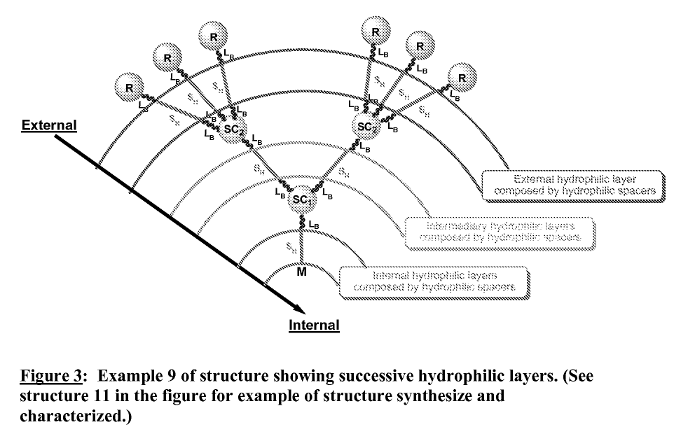

In the dentrimer structure of FIG. 3, wherein M, SC, R, L_{B} and S_{H} are as defined above, the hydrophilic layer may be inserted between:
the central core molecule and the secondary core molecule with degradable chemical linkages to provide hydrophilic multiple layers; or
the central core molecule and the drug with degradable chemical linkages to provide a monolayer, i.e. no secondary core molecule between said central core molecule and the drugs; or
the secondary core molecule and the drug with degradable chemical linkages to provide hydrophilic mono or multiple layers.
The drug or biologically active substance that may be included in the drug-conjugates of the present invention include but are not limited to:

Any substance intended for diagnosis, cure, mitigation, treatment or prevention of disease in humans or animals,

Examples of biologically actives molecules include, but are not limited to, peptides, proteins, enzymes, small molecule drugs, dyes, lipids, nucleosides, . . . Classes of small molecules drugs that are suitable for use with the invention include, but are not limited to, antibiotics, fungicides, anti-viral agents, anti-inflammatory agents, anti-tumor agents, cardiovascular agents, ophthalmic drugs, dermatological drugs and mixtures thereof.

The four key elements presented above, i.e. central core (CC), secondary core (SC), hydrophilic chemically defined spacer (S_{H}) and drug or other biologically active substance (D), will be linked together and will allow for a multiplicity of chemical structures which could be customized depending on the drug delivery system (DDS) characteristics targeted.

Controlled drug delivery from such chemical structures can be achieved by variations of several parameters in the structure's “architecture”. These variations would define the final DDS properties:

Number of drugs present on the structure (periphery).

Dissymmetrical structure: relative drug position, nature of drugs.

Different chemical linkages within same structure.

Core.

Secondary core or cores.

Hydrophilic spacer.

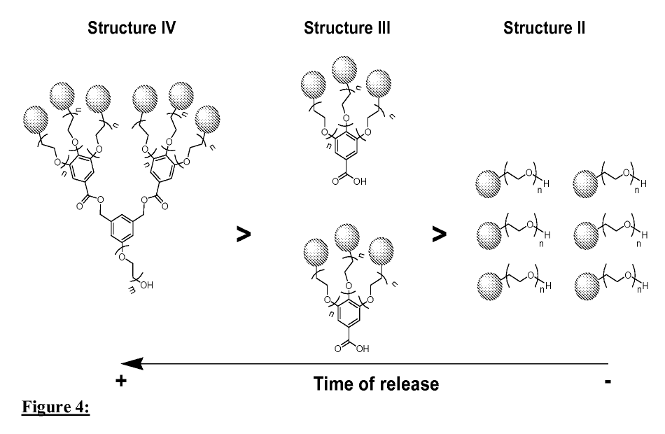

Preferably, the structures of this invention are built in such way each sub-unit or generation i.e. central core, secondary core, hydrophilic spacer and active ingredient are bonded to each other through an enzymatically degradable linkage. Assuming an equal proportion of the active ingredient is brought by each chemical system from structures II to IV, drug release in the body is expected to be respectively slower from structure IV of Figure compared to structures III and II of FIG. 4.

Structures containing different drug at different positions (internal or external) can be synthesized. (See FIG. 5, wherein CC, SC, M, L_{B}, D and S_{H} are as defined above.) Such structures can be symetrical or dissymetrical referring both to the drug position (internal or external) and to the drug nature. For example, the final structure may contain different active ingredients to enable polytherapy.

Structures containing sub-units attached by different enzymatically degradable links can be synthesized. Presence of such different chemical links implies different behavior in the presence of enzymes (difference in accessibility, in speed of cleavage . . . ) that inevitably induce differences in drug's release. (See FIG. 6, wherein CC, SC, M, L_{B}, D and S_{H} are as defined above.)

The invention is further illustrated by the following examples which are illustrative of a specific mode of practicing the invention and are not intended as limiting the scope of the claims.

### EXAMPLE

As previously described, different possibilities would allow defining a specific drug delivery system: the number of actives linked to the structure, their relative position to the central core (internal, external), the nature of the chemical links to the structure.

The dendrimer-drug conjugates of the present invention as shown in FIG. 4, are built in such way each sub-unit or generation i.e. central core, secondary core, hydrophilic spacer and drug or other biologically-active molecule is bonded to each an other through an enzymatically degradable linkage. Assuming an equal proportion of the drug is brought by each chemical system from structures II to IV, drug release in the body is expected to be respectively slower from structure IV compared to structures III and II.

Equal amounts of flurbiprofen bonded to structures II and III of FIG. 4 were incubated with esterases from pork liver. The enzymatic release of flurbiprofen was monitored using HPLC gradient method allowing both structures analysis as well as the elution of flurbiprofen.

While different products are expected from the enzymatic activity, the experiment was mainly focused on the detection and quantification of the initial structure (II and III) as well as on the apparition of flurbiprofen.

Structures II and III as well as flurbiprofen released from these two structures are quantitated using high performance liquid chromatography (HPLC). The analytes are eluted from a XTerra® Phenyl column using an eluent gradient composed of water/methanol/formic acid 50 mM. Analytes are detected by UV absorbance at 240 and 280 nm.

The results are shown in FIG. 7.

Structures II and III in quantities providing the same amount of flurbiprofen, were incubated with 25 UI of esterase at 37° C. The 25 UI esterase was daily renewed for a total of 4 days. Analysis was performed at times 0, 24 H, 48 H, 72 H and 96 hours after the addition of the enzyme.

The following results were obtained:

After four incubation days under conditions using the same quantities of esterase and flurbiprofen, about 70% of flurbiprofen base was released from structure II while only 10% was released from structure III. This difference in active drug release from the initial structure indicates that the active is cleaved from a simple structure, where all the bonds between the drug and the dendrimer are substantially identical, at a faster rate as compared to a complex structure wherein the bonds vary or the drug is present in different generations of the dendrimer. Furthermore, several peaks have been identified with the structure III that may correspond to intermediate products.

From structure IV, which comprises two Structure III units, covalently bonded together, the rate of dendrimer-drug conjugate having release of the active molecule would be slower. (See the results of this experiment in FIG. 8.)

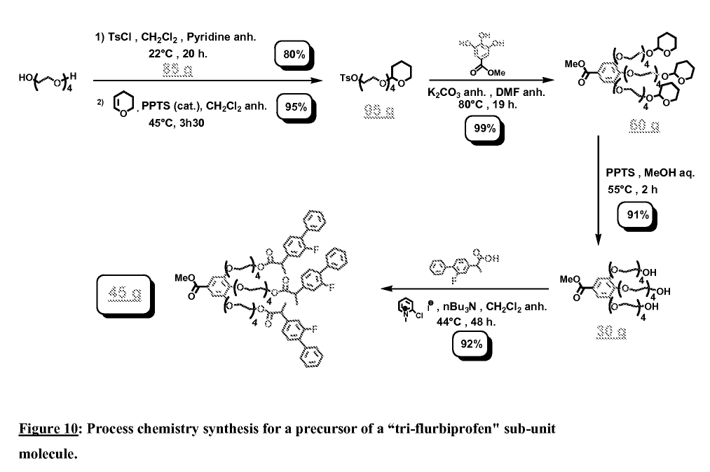

The dendrimer-drug conjugate having 3 flurbiprofen molecules bonded to a dendrimer molecule was successfully prepared by the process scheme set forth in FIG. 10. This conjugate was used in the above Example.

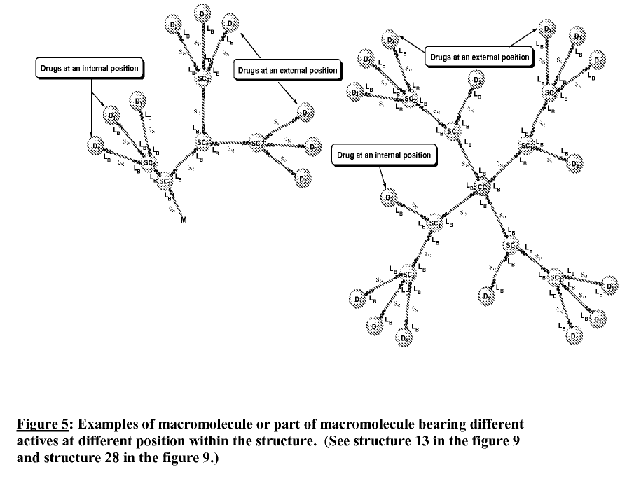

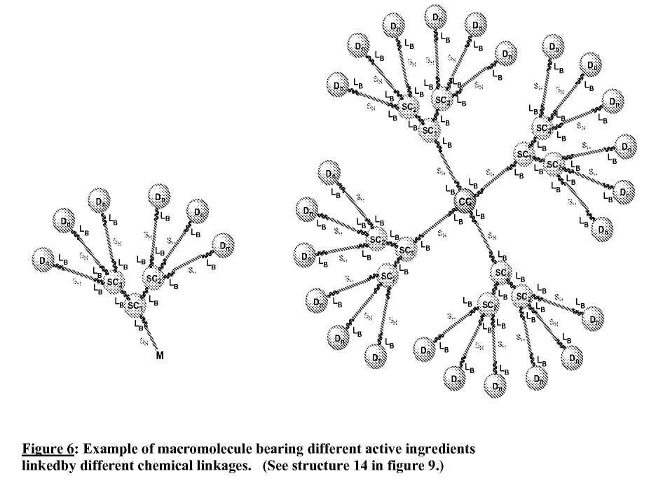

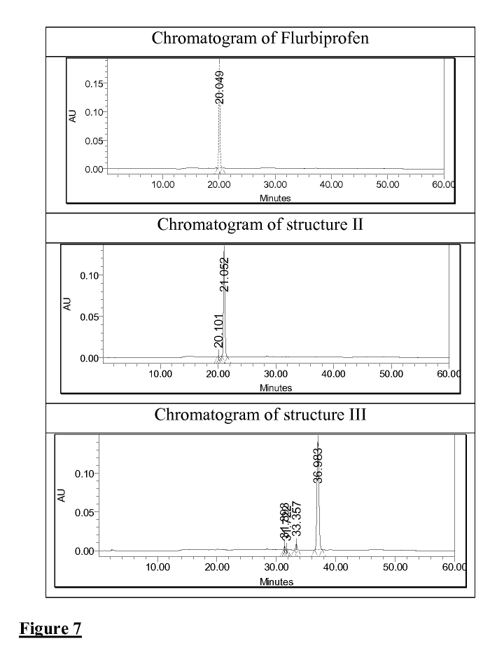

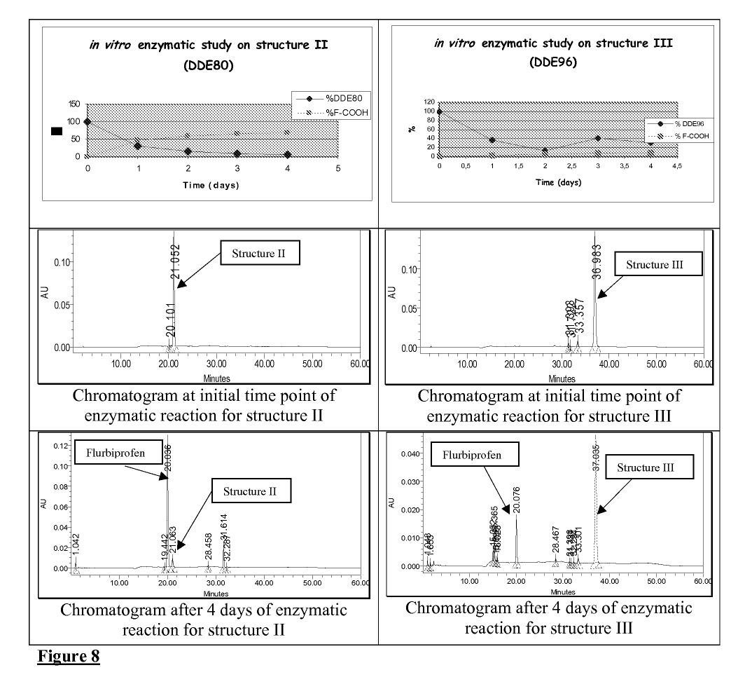

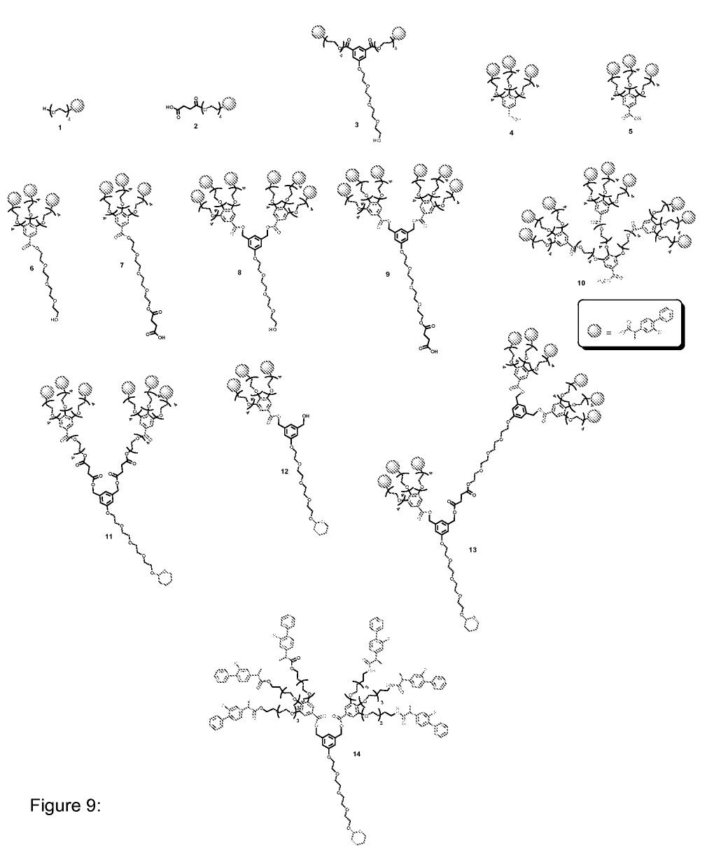

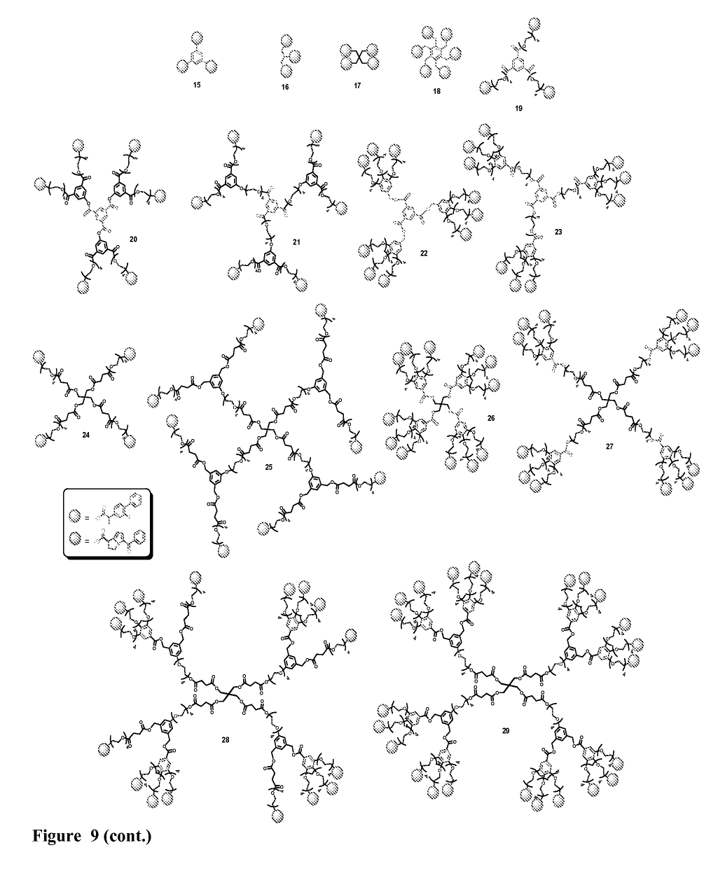

While particular embodiments of the invention have been described it will be understood of course that the invention is not limited thereto since many obvious modifications can be made and it is intended to include within this invention any such modifications as will fall within the scope of the appended claims. For example, as shown in FIG. 9, many variations on the dendrimer-drug conjugates described above may be made. Each of such variations, as shown in FIG. 9, are within the claimed scope of the invention.

1. A dendrimer-drug conjugate comprising the following elements;
(a) a central core molecule having at least two, and preferably three or more, functional groups capable of reacting with a first linear hydrophilic molecule to form a covalent bond between said central core molecule and said first linear hydrophilic molecule;
(b) a first linear hydrophilic molecule capable of reacting with said central core molecule to form a first segment of said conjugate comprising multiple branches of said first linear hydrophilic molecule emanating from said central core molecule, wherein said first segment of said conjugate comprises a covalent bond between said central core molecule and said first linear hydrophilic molecule and wherein said linear hydrophilic molecule comprises at least one additional functional group capable of reacting with a secondary core molecule to form a covalent bond between said first linear hydrophilic molecule and said secondary core molecule;
(c) a secondary core molecule having at least one functional group capable of reacting with said additional functional group of said first linear hydrophilic molecule to form a covalent bond between said secondary core molecule and said first linear hydrophilic molecule and having at least two additional functional groups capable of reacting with a second linear hydrophilic molecule;
(d) a second linear hydrophilic molecule having a functional group capable of reacting with said additional functional groups of said secondary core molecule to form a second segment of said conjugate comprising multiple branches of said second linear hydrophilic molecule emanating from said secondary core molecule, wherein said second segment of said conjugate comprises a covalent bond between said secondary core molecule and said second linear hydrophilic molecule and wherein said second linear hydrophilic molecule has at least one additional functional group capable of reacting with a drug or other biologically-active molecule; and
(e) one or more drugs or other biologically active molecules capable of reacting with said additional functional group of said second linear hydrophilic molecule to provide said dendrimer-drug conjugate.

2. A conjugate according to claim 1 wherein said covalent bond of element (d) is hydrolyzed in the presence of an endogenous esterase.

3. A conjugate according to claim 2 wherein said covalent bond is selected from the group consisting of an ester, an amide, a carbonate, a carbamate, a urea and mixtures thereof.

4. A conjugate according to claim 1 wherein either or both of said first linear hydrophilic molecule or said second linear hydrophilic molecule is a polyoxyethylene molecule.

5. A conjugate according to claim 1 wherein either or both of said central core molecule and said second core molecule is a polyhydroxy organic compound.

6. A conjugate according to claim 5 wherein either or both of said central core molecule and said secondary core molecule selected from the group consisting of isophthallic acid, gallic acid and derivatives thereof.

7. A conjugate according to claim 1 wherein said drug is selected from the group consisting of peptides, proteins, enzymes, small molecule drugs, dyes, lipids, nucleosides and mixtures thereof.

8. A conjugate according to claim 7 wherein said drug is a small molecule drug selected from the group consisting of antibiotics, fungicides, anti-viral agents, anti-inflammatory agents, anti-tumor agents, cardiovascular agents, ophthalmic drugs, dermatological drugs and mixtures thereof.

9. A conjugate according to claim 1 wherein the dendrimer further comprises a segment comprising a second secondary core molecule and a third linear hydrophilic molecule inserted between said first secondary core molecule and said biologically-active molecule.

10. A conjugate according to claim 9 wherein said drug-dendrimer conjugate comprises two or more different biologically-active molecules, including a first biologically active molecule bonded to said first secondary core molecule and a second biologically active molecule bonded to said second secondary core molecule.

11. A conjugate according to claim 1 or claim 9 wherein any of said linear hydrophilic molecules are replaced with a linear hydrophobic molecule.

12. A conjugate according to claim 1 or claim 9 wherein any of said linear hydrophilic molecules are replaced with a branched hydrophilic molecule or a linear or branched hydrophobic molecule.

13. A conjugate according to claim 1 or 9 wherein said central core molecule has three or more of said functional groups.

14. A drug-dendrimer conjugate comprising a central core, two or more linear hydrophilic molecules bonded thereto, a secondary core molecule bonded to a majority of said first linear hydrophilic molecules, a drug or other biologically-active molecule bonded to the remainder of said first linear hydrophilic molecules or bonded to a segment comprising a second secondary core molecule which is bonded to said first secondary core molecule by a linear hydrophilic molecule, and wherein at least some of the bonds between said drug or other biologically active molecule and said first and/or second secondary core molecules are hydrolysable by an endogenous esterase.

15. A drug-dendrimer conjugate comprising a central core molecule, two or more secondary core molecules bonded to said central core molecule, two or more linear hydrophilic molecules bonded to said secondary core molecules; a drug or other biologically-active molecule bonded to at least some of said first, linear hydrophilic molecules or bonded to a segment comprising a second secondary core molecule, bonded to said first secondary core molecule by a linear, hydrophilic molecule, wherein at least some of the bonds between said drug or other biologically active molecules and said first and/or second secondary core molecules are hydrolysable by an endogenous esterase.

16. A drug-dendrimer conjugate comprising a central core molecule, two or more linear hydrophilic molecules bonded thereto, a secondary core molecule bonded to a majority of said first linear hydrophilic molecules, a drug or other biologically-active molecule bonded to the remainder of said first linear hydrophilic molecules or bonded to a segment comprising a second secondary core molecule which is bonded to said first secondary core molecule by a covalent bond, wherein at least some of the bonds between said drug or other biologically active molecules and said first and/or second secondary cores are hydrolysable by an endogenous esterase.
